(19) United States
(12) Patent Application Publication
(10) Pub. No.: US 2010/0184177 A1
(43) Pub. Date: Jul. 22, 2010

(54) PLASTIC DISPOSABLE REACTOR SYSTEM

(75) Inventor: ANDREW G. MITCHELL, CHARDON, OH (US)

Correspondence Address: Emerson, Thomson & Bennett, LLC, 777 W. Market Street, Akron, OH, 44303, US

(73) Assignee: ENERGETIX, LLC, CHARDON, OH (US)

(21) Appl. No.: 12/692,038

(22) Filed: Jan. 22, 2010

(30) Foreign Application Priority Data
Jan. 22, 2009 (ZA) 2009/00499

(51) Int. Cl. C12P 5/00; C12M 1/00

(52) U.S. Cl. 435/166; 435/293.1; 435/292.1

(57) ABSTRACT

A plastic, disposable reactor (“PDR”) system is presented that will allow growth of microorganisms at various temperatures and pressures cost effectively. In this invention, the use of the system for aquaculture of algae is presented. The use of the reactor will allow carbon sequestration and significant production of a renewable energy source. The incorporation of recycled materials in various components of the plant also benefits the environment.

### I. FIELD OF THE INVENTION

This application claims priority to South African Patent Application No. 2009/00499 filed Jan. 22, 2009, which issued as ZA Patent No. 2009/00499 on Sep. 30, 2009, and incorporated herein by reference in its entirety. The invention is a plastic disposable reactor “PDR” system that can be employed in a variety of applications including but not limited to photobiosynthesis. The use of the PDR system in the cultivation of algae and the process associated therewith is incorporated in the present invention.

### II. BACKGROUND

The sequestration of carbon has received much recent attention and papers discussing algae aquaculture as a viable method have been published extensively. So has the treatment of wastewater using aerobic and anaerobic photobioreactors. Patents and other papers on both topics have been summarized by Elefritz et al (U.S. Pat. No. 7,455,765). A particular aspect of the papers focuses on the types of organisms incorporated, for example Kodo et al (U.S. Pat. No. 6,083,740) discuss the use of Spirulina as a viable organism. Wexler et al (U.S. Pat. Nos. 6,416,993 and 6,465,240) discuss the use of chlorella for treating a waste stream that has been neutralized by other prokaryotes and non sulphur bacteria.

In the growth of phototropic organisms one of the challenges is to present sufficient light to the organisms for maximum growth with the aim of as close to uniform light intensity throughout the support media (usually nutrient rich water). One approach, that of introducing light reflectors into the media of similar density, was reported by Arnaud Muller Feuga (U.S. Pat. No. 6,492,149). Other approaches have been related to the geometries of the reactor design. (Hoeksema U.S. Pat. No. 5,162,051; Robinson and Morrison U.S. Pat. No. 5,137,828; Trosh et al, U.S. Pat. No. 6,509,188). Later patents such as McCall's (US Patent Appln. No. 2008/0268302) disclose the use of edge illuminated light transmitting media such as acrylate for these purposes. Goldman et al (US Patent Appln. No. 2008/0293132) report the use of solar reflectors to concentrate light on a photobioreactor.

Other patents have reported other processes—Bayless et al, (U.S. Pat. No. 6,667,171) for example, patented a membrane process on which cyanobacteria and algae are supported. Cote & Behmann (U.S. Pat. No. 7,459,076) disclose a flow through granulator—a modified CSTR with aerobic and anoxic zones and an airlift pump. These generally employ algae of various types and certain bacteria such as cyanobacteria with or without solid or membrane support material in an aqueous media in housings which permit the penetration of light to support photosynthesis.

One of the intrinsic difficulties associated with the cultivation of algae is to keep the surfaces of the reactor vessels and internal components clean. Numerous patents have reported methods of incorporating cleaning mechanisms. For example, a method for controlling membrane fouling was reported by Hong et al, (U.S. Pat. No. 7,459,083). However the practicality and usefulness of these methods vary considerably. An interesting approach that has been developed is cited by Selker et al (US Patent Appln. No. 2008/0274541) who describe a disposable bag on a rocker that provides agitation by a wave motion.

Lewnard et al (US Patent Appln. No. 2008/0178739) provide a review of both open and closed system designs as well as a hybrid method for cultivating algae in large closed spaces. The main issues cited by most authors are the propensity for contamination in open systems as well as a fairly low yield in terms of algal growth per unit land area compared to closed systems, which have the associated comparative high capital cost per unit of land area. Closed systems have the advantage of increased carbon dioxide availability. Freeman (US Patent Appln. No. 2008/0254529) describes a process whereby liquid culture mediums are exposed to closed carbon dioxide/air mixtures. Whitton (US Patent Appln. No. 2008/0286851) describes a flexible integrated closed system constructed of thin plastics which can potentially be folded up and transported to different sites or mounted on earthen bearms. The inclusion of gas spargers is discussed. Howard et al (US Patent Appln. No. 2008/0299643) disclose a variant on the hybrid open/closed system with plastic pond covers and the introduction of diffused CO₂.

### III. SUMMARY

In the generation of biogas from wastewater plant digestate, cattle manure, or animal wastes, either by dry fermentation or wet anaerobic digestion, a methane rich gas containing typically 30% to 35% carbon dioxide is formed. Alternatively, carbon dioxide is produced in the combustion of hydrocarbons and the resulting exhaust gas typically contains 10% to 15% carbon dioxide.

In the process described, carbon dioxide containing gas is scrubbed with sufficient water under pressure to dissolve the carbon dioxide in a suitable gas liquid contacting device. One embodiment includes, but is not limited to, a tank, or series of tanks, filled with suitable support media (such as used plastic drinking bottle caps) through which water passes counter current to the treated gas. In this application, “tank” and “PDR” will be used interchangeably.

Carbon dioxide rich water is pumped to the PDR train, consisting of multiple units of the PDRs. The PDRs have been inoculated with and contain growing algae. The nutrient rich waters are fed upwards at low linear velocities through the PDRs and the resultant oxygen enriched water is drawn through a filter at the top of the PDR. The design of the filtration device and its fixture to the PDR is incorporated in this invention. In this embodiment, the linear velocity is between approximately 0 to approximately 0.01 m/s, which includes 0.001, 0.002, 0.003, 0.004, 0.005, 0.006, 0.007, 0.008, 0.009, and 0.010. In one embodiment the linear velocity is less than about 0.005 m/s.

The water is preheated to between about 24° C. and about 32° C. for optimal algae growth. (This temperature may change for other species of microbes).

The internal diameter of the PDR may vary from just greater than 0 to about 5 or more inches but is not limited to this upper limit.

The height of the PDR may vary from just greater than 0 to about 24 or more feet but is not limited to this upper limit.

The wall thickness of the PDR may vary from just greater than 0 to about ¼ inch or more but is not limited to this upper limit. The thickness of the reactor wall is determined by the design operating pressure, the internal diameter and height of the vessel using typical engineering considerations.

The inlet and exit of the PDR may have an internal pipe thread, an external pipe thread, or an external tube connector. This may be Imperial (BSP), metric (ISO), or US National Pipe Thread (NPT) and may be more or less than the typical 1 inch diameter.

The design of the PDR and the filtration device is incorporated in the invention.

The material of choice for the PDR for the purpose of aquaculture of algae is polyethylene teraphthalate (PET); however the PDR may be made of other suitable materials including, but not limited to, clear polyvinyl chloride (PVC), Polypropylene (PP), polyethylene (PE), high density polyethylene (HDPE), cross linked polyethylene (PEX), clear polycarbonate and other plastics.

### IV. DEFINITIONS

Biogas—a gas produced by the biological breakdown of organic matter in the absence of oxygen.

Plastic—any of various organic compounds produced by polymerization, capable of being molded, extruded, cast into various shapes and films, or drawn into filaments used as textile fibers.

Reactor train—at least two connected reactors.

### V. BRIEF DESCRIPTION OF THE DRAWINGS

Further features, benefits and advantages of the invention will become evident from the following description of exemplary embodiments with reference to the drawings, in which:

FIG. 1 shows a process flow diagram for the removal of carbon dioxide from a carbon dioxide rich stream and subsequent treatment of the carbon dioxide saturated or partially saturated water in two trains of PDRs;

FIG. 2 shows a detailed cross section of a PDR; and,

FIG. 3 shows a schematic of a PDR train.

### VI. DETAILED DESCRIPTION

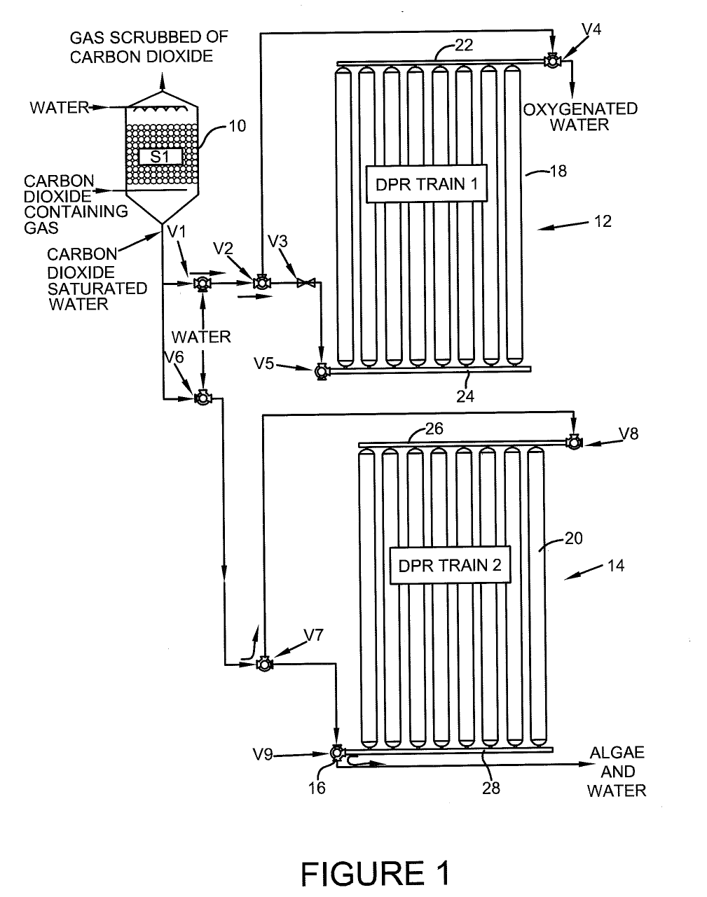

FIG. 1 shows at least one embodiment of a plant layout which removes carbon dioxide from an incoming gaseous stream by dissolution in water at ambient or elevated temperature and pressure. The carbon dioxide rich water stream 10 is conveyed through a series of three way ball valves V1, V2, V4, V5, V6, V7, V8, V9 (all valves with the exception of valve V3 which is a flow control valve) to the PDR units 18, 20. FIG. 1 shows the first PDR train 12, having a top fluid conveying pipe 22, bottom fluid conveying pipe 24, algae and water outlet 16, and PDRs 18. It also shows the second PDR train 14, having a top fluid conveying pipe 26, bottom fluid conveying pipe 28, and PDRs 20. In train 12 the valves V1, V2, V3, V5 are configured to allow the carbon dioxide rich water stream to pass upwards through the PDR train 12 containing algae. The algae in the course of photosynthetic metabolism convert the carbon dioxide to various complex organic molecules and oxygen. The oxygen (dissolved and gaseous) is conveyed from the algae by the continued upward motion of the water. In the second PDR train 14, the valves V6, V7, V9 are configured such that potable water is fed to the top of the PDR train allowing water and algae to be drawn from the bottom fluid conveying pipe 28 of the train and “harvested.” Once a fraction (in one embodiment, but not limited to, about one-half) of the algae has thus been withdrawn from each PDR 18, 20, the valves are reconfigured to allow either carbon dioxide enriched water or potable water (depending on the light cycle—i.e. either day or night) up through the PDR 18, 20.

Carbon dioxide rich water is pumped to the PDR train 12, 14, consisting of multiple PDRs 18, 20. The PDRs have been inoculated with and contain growing algae. The nutrient rich waters are fed upwards at low linear velocities through the PDRs and the resultant oxygen enriched water is drawn through a filter at the top of the PDR. The design of the filtration device 22 and its fixture to the PDR is incorporated in this invention.

The water is preheated to between about 24° C. and about 32° C. for optimal algae growth. (This temperature may change for other species of microbes). The internal diameter of the PDR may vary from just greater than 0 to about 5 or more inches but is not limited to this upper limit. The height of the PDR may vary from just greater than 0 to about 24 or more feet but is not limited to this upper limit. The wall thickness of the PDR may vary from just greater than 0 to about ¼ inch or more but is not limited to this upper limit. The thickness of the reactor wall is determined by the design operating pressure, the internal diameter and height of the vessel using typical engineering considerations. The inlet 56 and exit 54 of the PDR 38 may have an internal pipe thread 32, an external pipe thread 30, or an external tube connector 36. This may be Imperial (BSP), metric (ISO), or US National Pipe Thread (NPT) and may be more or less than the typical 1 inch diameter. The material of choice for the PDR for the purpose of aquaculture of algae is polyethylene teraphthalate (PET); however the PDR may be made of other suitable materials including, but not limited to, clear polyvinyl chloride (PVC), Polypropylene (PP), polyethylene (PE), high density polyethylene (HDPE), cross linked polyethylene (PEX), clear polycarbonate and other plastics.

A further embodiment of the described operation allows for the use of a bleaching agent in conjunction with potable water to clean the interior surface of the PDRs. Once this cycle has been completed, the cleaned PDRs will have to be re-inoculated with growing algae. This cleaning is helpful for continued maximum availability of light throughout the PDR.

After a period of time has elapsed, wherein the reactors may need to be replaced, the reactors are disconnected from the train and replaced with new reactors. The old reactors may be washed and sent for recycling.

The number of PDRs in a train and the number of trains employed for any given site will depend on various factors including, but not limited to, the quantity of gas to be treated, the availability of land space, the size distribution of the PDR units and the climatic conditions where the facility is to be situated.

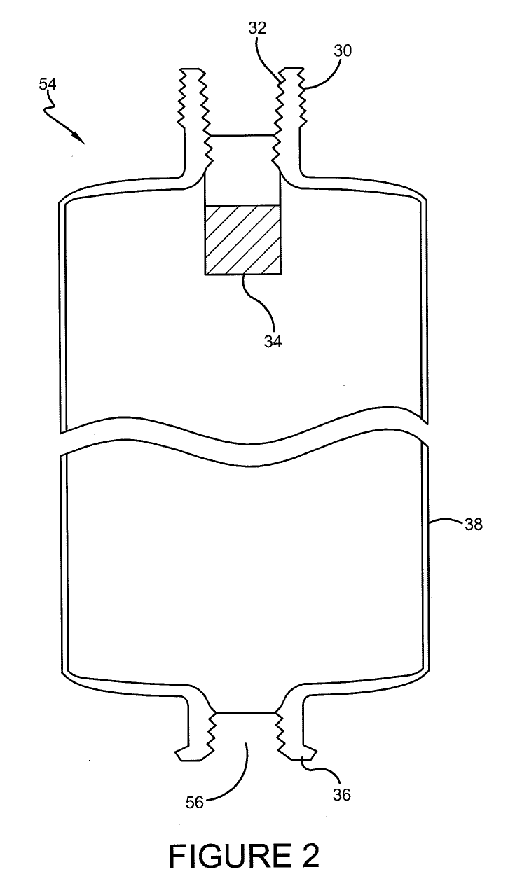

FIG. 2 shows one embodiment of a PDR 38 with the filtration mechanism 34 attached. The design of the PDRs has been discussed in the summary. The filtration device 34 is the counterpart of the female pipe thread—a male threaded fitting. The fitting incorporates a porous filtration medium 34 in the shape of a plug that is affixed to the tube. The bottom of the PDR 38 is affixed to the fluid conveying pipe 24, 28 by means of a suitable sized male threaded connection 36 and flexible hose.

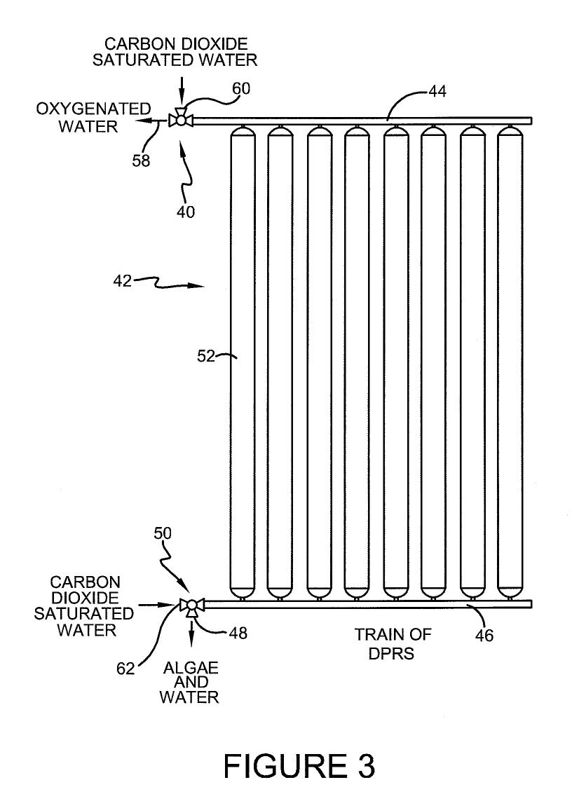

FIG. 3 shows one embodiment of a series of connected PDRs 52 forming a train 42. In the embodiment, these trains 42 will be suspended from an external support which attaches to the top water conveying pipe 44. FIG. 3 also shows valves 40, 50, oxygenated water output 58, carbon dioxide saturated water inlet 60, bottom carbon dioxide saturated water inlet 62, and algae and water outlet 48.

The above examples have been depicted solely for the purpose of exemplification and are not intended to restrict the scope or embodiments of the invention. The invention is further illustrated with reference to the claims that follow thereto.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The invention has been described with reference to several embodiments. Obviously, modifications and alterations will occur to others upon a reading and understanding of the specification. It is intended by applicant to include all such modifications and alterations insofar as they come within the scope of the appended claims or the equivalents thereof.

Having thus described the invention, it is now claimed:

What is claimed is:

1. A method for processing biogas, wherein the biogas contains carbon dioxide, the method comprising the steps of:
providing a reactor train, wherein the reactor train comprises at least two cylindrical plastic tanks, wherein the tanks contain algae;
dissolving the carbon dioxide into water;
moving the carbon dioxide saturated water through multiple valves, wherein at least one of the valves is operatively connected to a fluid conveying pipe, wherein the pipe is operatively connected to the plastic tanks; and,
converting the carbon dioxide to organic molecules and oxygen by moving the carbon dioxide saturated water through the algae.

2. The method of claim 1, wherein the method further comprises the steps of:
providing at least a second reactor train, the second reactor train comprising at least two cylindrical plastic tanks, the second reactor train having a top fluid conveying pipe and a bottom fluid conveying pipe;
drawing algae and water from the bottom pipe through the tanks; and,
when at least approximately one half of the algae has been drawn into the tanks, reconfiguring at least two valves to draw additional carbon dioxide saturated water through the tanks; and,
converting the carbon dioxide to organic molecules and oxygen.

3. The method of claim 1, wherein the method further comprises the steps of:
cleaning the interior of the tanks with a bleaching agent and water; and,
placing algae in the cleaned tanks.

4. The method of claim 2, wherein the method further comprises the steps of:
cleaning the interior of the tanks with a bleaching agent and water; and,
placing algae in the cleaned tanks.

5. The method of claim 3, wherein the tanks are made of a material chosen from the group comprising: polyethylene teraphthalate, clear polyvinyl chloride, polypropylene, polyethylene, high density polyethylene, cross-linked polyethylene, and clear polycarbonate.

6. The method of claim 4, wherein the tanks are made of a material chosen from the group comprising: polyethylene teraphthalate, clear polyvinyl chloride, polypropylene, polyethylene, high density polyethylene, cross-linked polyethylene, and clear polycarbonate.

7. The method of claim 6, wherein the tanks are made of polyethylene teraphthalate.

8. The method of claim 7, wherein the water is preheated to between about 24° C. and about 32° C., wherein the carbon dioxide saturated water is moved through the algae at a linear velocity of between approximately 0 m/s to approximately 0.01 m/s.

9. A plastic reactor system, wherein the system comprises:
a gas-liquid contacting device;
a top fluid conveying pipe;
a bottom fluid conveying pipe; and,
at least two plastic tanks, the tanks being operatively attached to the conveying pipes, the tanks containing algae.

10. The system of claim 9, wherein the tanks are made of a material chosen from the group comprising: polyethylene teraphthalate, clear polyvinyl chloride, polypropylene, polyethylene, high density polyethylene, cross-linked polyethylene, and clear polycarbonate.

11. The system of claim 9, wherein the system further comprises:
at least a second reactor train, the second reactor train comprising at least two cylindrical plastic tanks, the second reactor train having a top fluid conveying pipe and a bottom fluid conveying pipe.

12. The system of claim 10, wherein the system further comprises:
at least a second reactor train, the second reactor train comprising at least two cylindrical plastic tanks, the second reactor train having a top fluid conveying pipe and a bottom fluid conveying pipe.

13. The system of claim 11, wherein the tanks comprise:
a filter; and,
a connection device comprising a male to male connector with a tube insert welded to the connector and attached to a plug of porous plastic material of diameter less than a nominal thread diameter of the connector.

14. The system of claim 12, wherein the tanks comprise:
a filter; and,
a connection device comprising a male to male connector with a tube insert welded to the connector and attached to a plug of porous plastic material of diameter less than a nominal thread diameter of the connector.

15. The system of claim 13, wherein the tanks have an internal diameter, a height, and a wall thickness, wherein the system further comprises:
the internal diameter is between about 0 and about 5 inches, the height is between about 0 and about 24 feet, and the wall thickness is between about 0 and about ¼ inch.

16. The system of claim 14, wherein the tanks have an internal diameter, a height, and a wall thickness, wherein the system further comprises:
the internal diameter is between about 0 and about 5 inches, the height is between about 0 and about 24 feet, and the wall thickness is between about 0 and about ¼ inch.

17. The system of claim 13, wherein the tanks have an internal diameter, a height, and a wall thickness, wherein the system further comprises:
the internal diameter is greater than about 5 inches, the height is greater than about 24 feet, and the wall thickness is greater than about ¼ inch.

18. The system of claim 14, wherein the tanks have an internal diameter, a height, and a wall thickness, wherein the system further comprises:
the internal diameter is greater than about 5 inches, the height is greater than about 24 feet, and the wall thickness is greater than about ¼ inch.

19. A method for processing biogas, wherein the biogas contains carbon dioxide, the method comprising the steps of:
providing a reactor train, wherein the reactor train comprises at least two cylindrical plastic tanks, wherein the tanks contain algae;
dissolving the carbon dioxide into a liquid media;
moving the carbon dioxide saturated liquid media through multiple valves, wherein at least one of the valves is operatively connected to fluid conveying pipe, wherein the pipe is operatively connected to the plastic tanks; and,
converting the carbon dioxide to organic molecules and oxygen by moving the carbon dioxide saturated liquid media through the algae.

20. The method of claim 19, wherein the method further comprises the step of:
extracting an oxygen enriched stream.
